(19) United States
(12) Patent Application Publication
(10) Pub. No.: US 2025/0098997 A1
(43) Pub. Date: Mar. 27, 2025

(54) METHOD AND SYSTEM OF IDENTIFYING, NEUTRALIZING, AND REPLACING NEGATIVE BELIEF SYSTEMS WITHIN THE SUBCONSCIOUS MIND

(71) Applicant: Nordyke Business Associates, LLC, Carson City, NV (US)

(72) Inventors: Bradley Brian Nelson, St. George, UT (US); Jean Kimberly Nelson, St. Geoge, UT (US); Joshua Dale Nelson, Cedar City, UT (US); Susanne Hufnagel, Herzogenaurach (DE)

(73) Assignee: Nordyke Business Associates, LLC, Carson City, NV (US)

(21) Appl. No.: 18/628,502

(22) Filed: Apr. 5, 2024

Related U.S. Application Data
(60) Provisional application No. 63/585,271, filed on Sep. 26, 2023.

(51) Int. Cl. A61B 5/16; A61B 5/00

(52) U.S. Cl. CPC A61B 5/165; A61B 5/4519

(57) ABSTRACT

A method of identifying negative beliefs within the subconscious mind includes presenting a mind map to a subject, asking the subject a series of questions based on the mind map, administering muscle response testing, and identifying a negative belief based on the muscle response testing. The method may further include removing the negative belief and installing a positive belief by swiping a magnetic device over the subject's governing meridian while holding the intention to release the negative belief and install the positive belief. In some examples, the method includes using a monitoring device having sensors and software designed to measure physiological responses.

### CROSS-REFERENCE TO RELATED APPLICATIONS

This application claims the benefit of U.S. Provisional Application Ser. No. 63/585,271, filed on Sep. 26, 2023, which is incorporated herein by reference.

### TECHNICAL FIELD

The present disclosure relates to subconscious belief systems and their association with mental health imbalances. More particularly, the present disclosure relates to muscle response testing protocols using a mind map to identify negative beliefs within the subconscious mind and replace them with positive beliefs.

### BACKGROUND

It is estimated that the subconscious mind comprises around 90-96% of human intelligence. Negative belief systems within the subconscious mind, however, can hinder an individual's progress and well-being. The fields of psychology and self-improvement have long struggled to address the consequences of negative belief systems deeply entrenched within an individual's subconscious mind. Traditional therapeutic approaches have limitations in precisely identifying and effectively neutralizing these negative belief systems, which limits the progress of individuals seeking personal growth and mental health.

Some approaches to understanding subconscious beliefs include: psychoanalysis, cognitive behavioral therapy, hypnotherapy, neuro-linguistic programming, mindfulness and meditation, positive psychology, self-help books and seminars, energy healing, positive affirmations, and life coaching. Still, each of these approaches are not without their challenges and disadvantages. For example, there is significant variation in efficacy amongst individuals, and determining the right approach can be a process of trial-and-error. In addition, many therapeutic approaches, such as psychoanalysis and traditional talk therapy, can be time-consuming and require long-term commitment from the patient. This can be a barrier for individuals seeking quicker results. Professional therapy and coaching can likewise be expensive, making them inaccessible to some individuals. Other approaches, like hypnotherapy or energy healing, require the involvement of practitioners, and their effectiveness can vary based on the skill and expertise of the practitioner. Negative beliefs can also be deeply ingrained, and individuals may resist changing them, even when they recognize their harmful effects, thereby hindering progress.

In addition, while promoting positive thinking is important, some self-help approaches oversimplify the process and imply that merely thinking positively is enough to overcome deep-seated negative beliefs. Some self-help literature and motivational speakers may further set unrealistic expectations for personal growth and transformation, leading to disappointment when quick and dramatic changes do not occur. Even when individuals successfully address negative beliefs, they may still face relapses or moments of self-doubt, as old thought patterns can resurface during times of stress or challenge. Some approaches may not take into account cultural or individual differences, which can limit their applicability and effectiveness for diverse populations. In some cases, individuals may be vulnerable to unethical practices or exploitation, especially in the self-help industry. Lastly, our understanding of the subconscious mind and the mechanisms behind negative beliefs are still evolving. As a result, some approaches may not fully grasp the complexity of the issue.

In U.S. Pat. No. 9,330,240, entitled a Method And System Of Identifying Bodily Imbalances, Applicant discussed the relationship between the subconscious mind and bodily ailments. The associations between the subconscious mind and one's belief systems, however, remain to be properly investigated. An individual's subconscious mind forms negative belief systems about the world, other people, and the self, for example, that can be detrimental to that person's mental wellbeing. These negative beliefs can also interfere with one's full exercise of their will, thereby limiting their ability to create the health, wealth, love, and happiness of which they are capable. As such, there is a need in alternative medicine for a system that guides a practitioner when using muscle response testing to identify faulty programming amongst one's belief systems that can limit mental and emotional wellbeing. The current method and system seek to solve these problems and others, thereby improving an individual's mental and emotional wellbeing.

### SUMMARY OF EXAMPLE EMBODIMENTS

A method of identifying negative beliefs within the subconscious mind comprises presenting a mind map to a subject, asking the subject a series of questions related to the mind map, performing muscle response testing, and identifying a negative belief based on the muscle response testing. The mind map comprises one or more categories, one or more subcategories, and one or more sets of negative beliefs. The categories of the mind map, in some embodiments, comprise Divine, Communication, Power, Survival, Insight, Love, and Passion.

The method of identifying negative beliefs within the subconscious mind may further comprise removing the negative belief by swiping a magnetic device, such as a magnet or the tester's hand, over the subject's governing meridian while holding the intention to release the negative belief. The method of identifying negative beliefs within the subconscious mind may further comprise installing a positive belief thereafter by swiping a magnetic device over the subject's governing meridian while holding the intention to install the positive belief.

### BRIEF DESCRIPTION OF THE DRAWINGS

FIG. 1 illustrates an example embodiment of the general categories of a mind map;

FIG. 2 illustrates an example embodiment of a subcategory of a mind map;

FIG. 3 illustrates an example embodiment of a set of negative beliefs of a mind map;

FIG. 4 illustrates an example flowchart of a method of identifying negative beliefs;

FIG. 5 illustrates an example flowchart of a method of identifying negative beliefs;

FIG. 6 illustrates an example flowchart of a method of removing negative beliefs; and

FIG. 7 illustrates an example flowchart of a method of installing positive beliefs.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.).

As used herein, the term “user” may be a physician (e.g., doctor, chiropractor), a holistic practitioner, a tester, a subject, or other person who uses the methods of the present disclosure to identify negative beliefs within themselves or others. The term “subject” is defined as the affected person seeking an identification of the negative beliefs within their subconscious mind. In some embodiments, the user may be the tester who administers the muscle response testing to the subject to identify a negative belief within the subject's subconscious mind. In other embodiments, the user may be both the tester and the subject when self-administering the muscle response testing to identify a negative belief within their own subconscious mind.

As previously discussed, there is a need for a process to guide a user through the administration of muscle response testing to identify faulty programming and negative beliefs held within a subject's subconscious mind which may be affecting mental and emotional wellbeing. Once identified, a need for a process further exists that enables the user to neutralize these negative, underlying self-beliefs and replace them with positive beliefs and affirmations. The present disclosure addresses these challenges and others.

In some embodiments, a method of identifying negative beliefs comprises a tester presenting a mind map to a subject, asking the subject one or more questions based on the mind map, performing muscle response testing to determine the answers to the questions, and identifying the negative belief held within the subject's subconscious mind. Once the negative belief is identified, the method may further comprise neutralizing the negative belief via a magnetic field device and subsequently implanting a positive belief in place thereof. It will be appreciated that neutralization and removal of the negative belief, followed by the implantation of the positive belief, together foster alignment between the subject's thoughts and actions, leading to improved mental wellbeing.

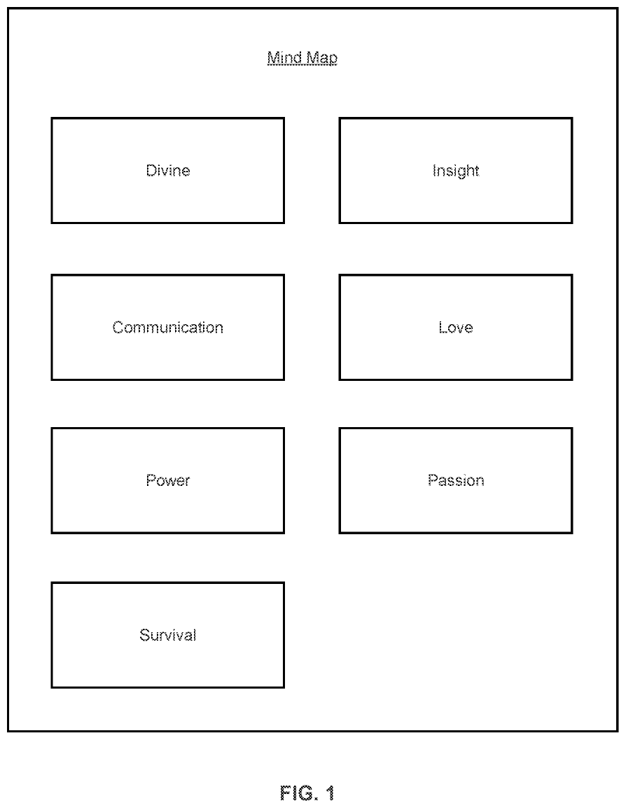

As shown in FIG. 1, a mind map may comprise a diagram or table whereby information is organized into a hierarchy. The mind map outlines relationships amongst individual negative beliefs to assist the user in mapping and understanding the user's overall belief system. The mind map may be presented in a printed form, shown via electronic means, or otherwise recited verbally to the subject. Without limitation, each page of the mind map may comprise information that is organized into a left column and a right column, with corresponding rows therein. During the administration of muscle response testing, the organization of the mind map quickly guides the tester towards the identification of the negative belief at issue within the subject.

In some embodiments, the mind map comprises seven (7) top-level categories: Divine, Communication, Power, Survival, Insight, Love, and Passion. While these categories have been given specific names herein, it will be appreciated that equivalent categories, regardless of nomenclature, are likewise contemplated. In other words, the categories may be further grouped and defined by their chakra connection, gland connection, muscle connection, system connection, and/or Maslow's Hierarchy of Needs. As the energy centers of the body, it will be appreciated that the seven chakras each correspond energetically to particular glands, muscles, and organ systems within the body. An understanding of these connections helps the tester to identify the negative belief held within the subconscious mind of the subject and offers valuable insight into the subject's subconscious thought patterns. The seven categories of the mind map and their connections map may be presented as outlined below:

### 1. Divine

Crown Chakra: Spirit, Divine, Self-Realization, Wisdom, Spirituality
Gland Connection: Pineal Gland
Muscle Connection: Muscles Of The Eye
System Connection: Central Nervous System, Integumentary System
Maslow's Hierarchy: Self-Actualization/Transcendence

### 2. Communication

Throat Chakra: Communication, Self-Expression, Purification, Learning
Gland Connection: Thyroid Gland
Muscle Connection: Teres Minor
System Connection: Respiratory System
Maslow's Hierarchy: Cognitive

### 3. Power

Solar Plexus Chakra: Self-Esteem, Confidence, Action, Identity, Power
Gland Connection: Adrenal Glands
Muscle Connection: Calf Muscles, Sartorius Muscles, And Gracilis Muscles
System Connection: Digestive, Muscular, And Microbiome Systems
Maslow's Hierarchy: Belonging/Love

### 4. Survival

Root Chakra: Security, Safety, Support, Survival
Gland Connection: Gonads, Testes/Ovaries
Muscle Connection: None Known
System Connection: Reproductive And Skeletal Systems
Maslow's Hierarchy: Physiological

### 5. Insight

Brow Chakra: Intuition, Intellect, Insight, Perception, Big Picture
Gland Connection: Hypothalamus And Pituitary Gland
Muscle Connection: None Known
System Connection: Autonomic Nervous System And Endocrine System
Maslow's Hierarchy: Aesthetic

### 6. Love

Heart Chakra: Compassion, Unconditional Love, Kindness
Gland Connection: Thymus Gland
Muscle Connection: Teres Major
System Connection: Circulatory, Immune, And Lymphatic Systems
Maslow's Hierarchy: Esteem

### 7. Passion

Sacral Chakra: Passion, Creativity, Pleasure, Trust, Sexuality
Gland Connection: Testes/Ovaries,
Muscle Connection: None Known
System Connection: Genitourinary System: Urinary And Reproductive Systems
Maslow's Hierarchy: Safety

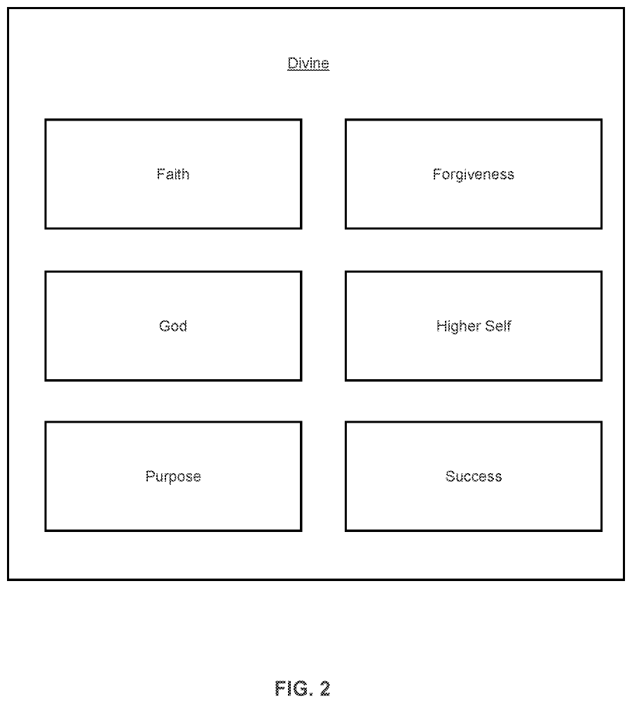

As shown in FIG. 2, each of the seven categories of the mind map further comprises a unique series of subcategories that guide the tester in an effort to promptly identify the negative belief at issue. For example, the Divine category contains the subcategories of Faith, Forgiveness, God, Higher Self, Purpose, and Success.

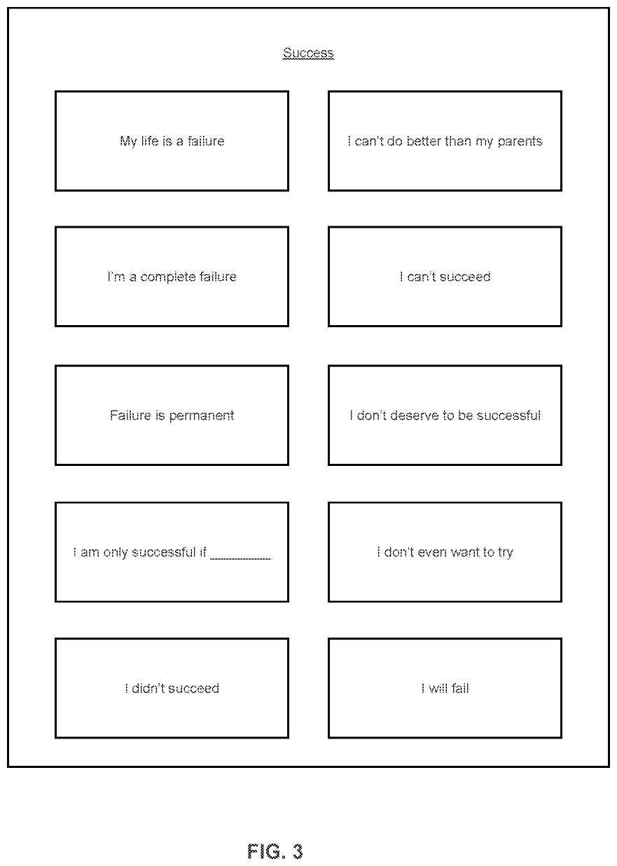

As shown in FIG. 3, each of the subcategories may further comprise a non-exhaustive set of negative beliefs that are thematically or emotionally related to one another. The subcategory of Success, in some embodiments, may comprise a set of negative beliefs such as “My life is a failure,” “I'm a complete failure,” “Failure is permanent,” “I am only successful if,” “I didn't succeed,” “I can't do better than my parents,” “I can't succeed,” “I don't deserve to be successful,” “I don't even want to try,” “I will fail,” etc. In practice, each set of negative beliefs is derived from experiential data obtained during treatment sessions.

Movement from the broader, top-level categories to the narrower subcategories represents diagnostic progression ultimately towards identification of the negative belief trapped within the subject's subconscious mind. In other words, once the proper category and subcategory have each been identified, the tester may next identify the negative belief at issue from the particular set of possible negative beliefs thereunder. While the mind map is described herein as having particular categories, subcategories, and sets of negative beliefs, the information as presented on the mind map may vary structurally without departing from the teachings herein.

Muscle response testing may be administered by the tester to ascertain answers to questions from the subject's subconscious mind. Muscle response testing, also known as applied kinesiology, is an alternative medicine practice that, in part, can be used to diagnose underlying mental and emotional ailments. In some embodiments, the tester asks the subject a “yes” or “no” question and then applies some physical pressure to the subject. In one example, the tester pushes the subject's extended arm down from a horizontal position, whereby resistance to the pushing indicates a “yes” response to the question and lack of resistance to the pushing indicates a “no” response. In similar fashion, in another example, the tester pulls a finger through a loop made by the interlocking of the thumbs and index fingers in order to ascertain an answer to the question from the subject's subconscious mind, whereby pulling the finger through the loop indicates a “no” response and failure to pull the finger through the loop indicates a “yes” response. Accordingly, the answer to the question asked is indicated by the relative strength or weakness of the indicator muscle being tested. While two methodologies for muscle response testing are described, multiple methods exist for testing a subject.

In some embodiments, the method comprises using a monitoring device having sensors and software configured to measure physiological responses, such as galvanic skin response, skin conductivity and temperature, breathing patterns, electrical brain activity, and/or heart rate variability. The monitoring device may be used by the tester to establish communication with the subconscious mind and independently verify the “yes” and “no” responses obtained via muscle response testing. This biofeedback provided by the monitoring device may be used alternatively or in addition to muscle response testing.

In some embodiments, the method may comprise using pendulum dowsing to ascertain answers to questions posed by the tester. In said embodiments, the tester holds a pendulum needle steady and then releases it upon asking a question regarding the mind map. Movement of the pendulum needle backwards and forwards may indicate a “yes” response while movement of the pendulum needle from side to side may indicate a “no” response. Alternatively, movement of the pendulum needle clockwise may indicate a “yes” response while movement of the pendulum needle counterclockwise may indicate a “no” response.

Biofeedback testing, whether using a monitoring device or pendulum, or through muscle response testing, leverages physiological markers to communicate with the subconscious mind. When the subconscious mind encounters a negative belief, subtle physiological changes occur, enabling the body to provide “yes” and “no” responses. These “yes” and “no” responses can be interpreted by the tester to facilitate the identification of negative beliefs within the subject's subconscious mind.

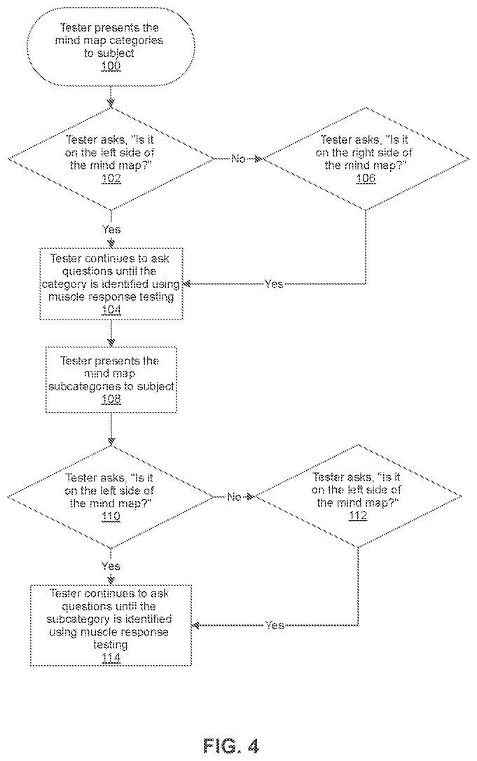

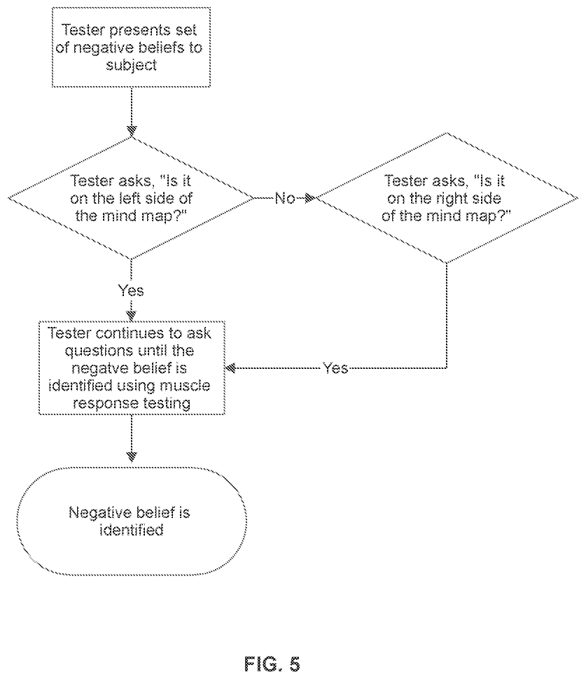

As shown in FIGS. 4-5, a method of identifying negative beliefs comprises the tester, at step 100, presenting a mind map (see FIG. 1) to the subject and asking, at step 102, “Is it on the left side of the mind map?” The tester then administers muscle response testing to determine the answer. The subconscious mind of the subject knows and is therefore able to identify, through muscle response testing, the category within which the negative belief is located. If a “yes” response is given by the indicator muscle, the tester then proceeds to ask the subject, at step 104, sequentially whether the negative belief is the first, second, third, etc. category on the left side of the mind map, until an identification of the category is reached. Alternatively, if a “no” response is given by the indicator muscle, the tester instead asks the subject, at step 106, “Is it on the right side of the mind map?” The tester then proceeds to ask the subject sequentially whether the negative belief is the first, second, third, etc. category on the right side of the mind map, until an identification of the category is reached. The tester next presents, at step 108, a mind map of the subcategories associated with the identified top-level category. The process continues as before, at steps 110 and 112 until the tester identifies, in step 114, the subcategory from the mind map indicated by muscle response testing.

For example, if the subconscious mind identifies the “Divine” category from FIG. 1, the tester is then presented with a series of subcategories illustrated generally at FIG. 2. The tester then determines, using muscle testing, in which of the subcategories the subject has the negative belief. Were the subconscious mind to identify the “Success” subcategory through muscle response testing, the tester would then present the set of negative beliefs at FIG. 3 associated thematically with that subcategory. The tester continues to ask questions of the subject until the particular negative belief at issue is identified from the larger set of possible negative beliefs.

Corresponding subcategories for Communication, Power, Survival, Insight, Love, and Passion with additional sets of negative beliefs are available to the tester under each subcategory (not depicted). It will be appreciated that varying arrangements, graphics, and nomenclatures may be used without departing from the present invention.

In some embodiments, the process of identifying negative beliefs may comprise determining the components and origins of the negative belief. The negative belief, for example, may comprise basic, split, parallel, tangled, or partial components. The negative belief may further comprise a limiting belief, faulty core belief, or faulty core identity component. The origin of each of these negative beliefs and their components may arise from the self, a suggestion, or otherwise be inherited from family. The components and origins of the identified negative belief can be assessed by muscle response testing, as outlined above and as shown in FIGS. 4 and 5 (i.e., asking the subject relevant questions about the mind map). After the tester decodes the entire belief system, the negative belief and its components and origins, he or she moves to any associations with the negative belief. Associations with the negative belief may comprise trapped emotions, images, broadcast messages, idea allergies, etc. If an association needs to be decoded, the tester returns to the general categories in FIG. 1 or to another mind map to decode and address said associations and any related imbalances.

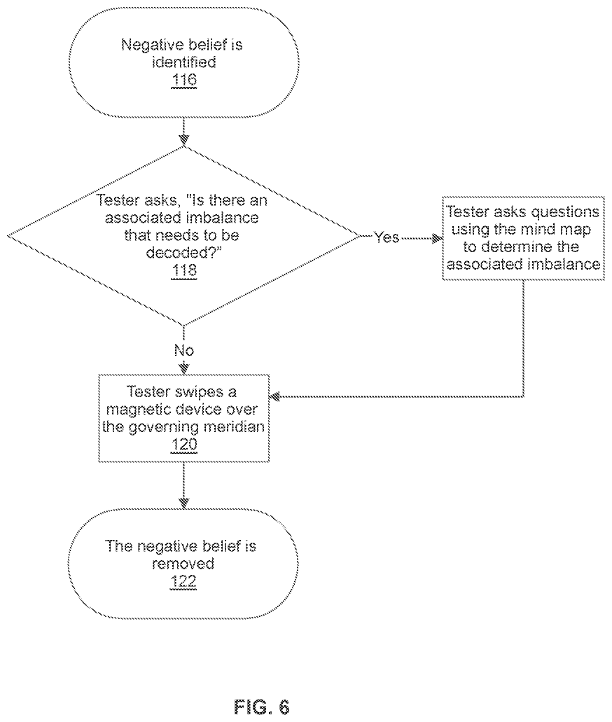

As shown in FIG. 6, once the negative belief system and any associations are identified through testing as described and shown in FIGS. 4-5, the tester then returns to the original faulty core belief, at step 116, and asks the subject, at step 118, “Is there an associated imbalance that needs to be decoded?” If the answer is “no,” the negative belief is now ready to be removed from the subject's subconscious mind. To remove the negative belief, the tester, at step 120, swipes three times over any length of the governing meridian with a magnetic device (e.g., a magnet or the tester's hand) while holding the intention to release the negative belief, releasing the negative belief at step 122. If the answer at step 118 was “yes,” the tester then asks questions using the mind map to determine the associated imbalance. Once the imbalance is identified, the method proceeds to steps 120 and 122.

The governing meridian begins at the base of the pelvis and travels up the back, over the top of the head to the lip, along the midline of the body. Use of a magnetic device or a magnetic field such as one's hand can release negative beliefs associated with energy stored along the governing meridian. It will be appreciated that the magnetic device utilizes electromagnetic principles such as resonance to interact with the governing meridian of the subject's body, thereby influencing energy flow and neutralizing the negative belief. The magnetic device may be of any polarity, modality, or strength that is safe for human use. In some embodiments, the tester may act as a proxy for the subject and swipe the magnet over themselves instead of the subject.

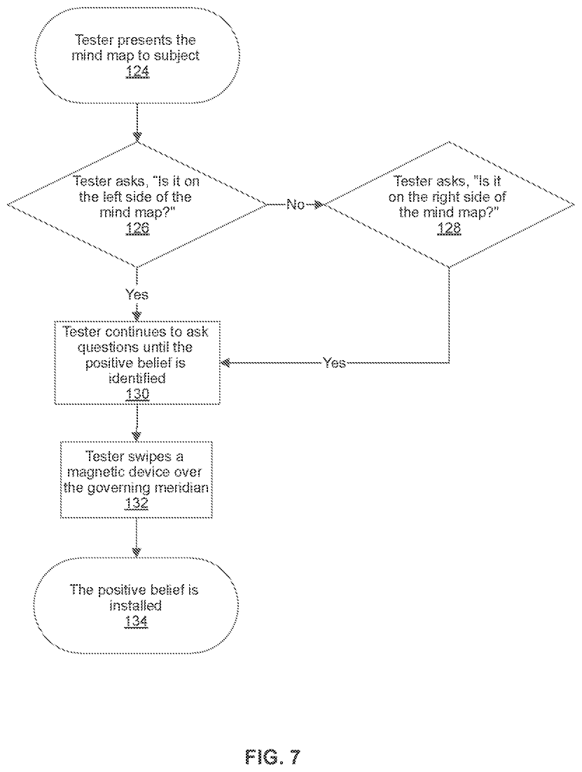

As shown in FIG. 7, in some embodiments, the method comprises installing positive replacement beliefs within the subject's subconscious mind. Positive beliefs that can be installed may comprise a positive program, a strengthening belief, an empowering core belief, or an empowering core identity. The appropriate positive belief to be installed may be identified from a set or list of positive beliefs using muscle response testing. To install the positive belief, the tester swipes the magnetic device three times over any length of the governing meridian while holding the intention to install the positive belief. Once installed, the positive belief fosters alignment between desired thought patterns and actions.

In one example, as shown in FIG. 7, a tester presents, at step 124, a mind map to a subject. The tester then asks, in steps 126 and/or 128, if the belief is on the left or right side of the map and will use muscle testing to determine the correct response (e.g., a firm response to muscle testing in response to the question in step 126 is a positive response and indicates that the left side of the map is correct). The tester repeats these steps in step 130 until the most beneficial positive belief is identified. Once the positive belief is identified, then at step 132, a user swipes a magnetic device over the governing meridian and the positive belief is installed to the user in step 134.

Beliefs, both positive and negative, are subjective views held by the subconscious mind that something is true or false, right or wrong, and act as the filters through which life is viewed and experienced. A subconscious belief exists as a distinct, singular pattern of energy and represents a global and absolute truth created by self-conclusion or through suggestion. Thus, only subconscious beliefs that are incongruent with conscious beliefs can be changed energetically. A person must have the desire to change an incongruent, subconscious belief, while a conscious belief cannot be changed except through conscious effort.

In one example of use of the methods described herein, a boy was adopted that had been with his adoptive family for about two years. The adoptive mother sought help because the boy exhibited behaviors such as overeating (even to the point of stealing food from others), constant lying, refusal to attach, and an unwillingness to learn. The boy's behaviors were believed to stem from his experiences before being adopted. Upon analysis using the method for identifying a negative belief, the following negative beliefs were identified:
Negative Program (NP1): “I have to get it before someone else.”
Negative Program (NP2): “I have to hide or use it before it is taken from me.”
Limiting Belief (LB1): “If I get caught, I'll be beat up.”
Limiting Belief (LB2): “I won't have enough.” (This was linked to a fear of survival.)
Faulty Core Belief (FCB): “I'm all alone; I have to fight for myself.”
Faulty Core Identity (FCI): “If I stay little, I don't have to fight for myself.”

These negative beliefs suggested that the boy felt he had to remain young and helpless to survive and get enough to eat. If he were to grow up or learn, he believed he would be moved to an older age group where he would be the smallest and most vulnerable in the group. After therapy sessions, the boy's mother reported significant improvements in his behavior. He began eating normally, bonded and cuddled with his family, started telling the truth, and began learning at grade level. Accordingly, the method and system of identifying, neutralizing, and replacing negative belief systems within the subconscious mind identified and released negative beliefs within the boy's subconscious that then enabled him to find healing from emotional and mental imbalances in his life.

In some embodiments, a mind map is presented using an electronic means for interacting with a series of links until a final identification is made. In said embodiment, the electronic means comprises a computer readable medium, such as a compact disc, ROM memory, or flash memory, that is inserted or otherwise installed onto a personal electronic device (e.g., smartphone or tablet), computer, or other computing device. For example, a physician with a computer or tablet in the examination room may either view the files electronically or may run an executable file that allows for installation on the computer, or that otherwise allows a user to interact with the mind map. The tester will then click on links, files, or folders to interact with the mind map and reach an identification. For example, a user will be shown the broad categories as illustrated in FIG. 1, such as via a tablet. A user will then select a category, such as by touching, clicking, double-clicking, or the equivalent, to reach the subcategories in FIG. 2 and the sets of negative beliefs in FIG. 3 until a final identification of the proper negative belief is reached.

In some embodiments, the electronic means comprises hosting the media on a 3^{rd} party location (e.g., a server or website), wherein the user may connect to the server using an internet-enable device such as a personal computer, tablet, smartphone, etc. to interact with the links. In other embodiments, the tester may perform the methods as taught in this disclosure remotely over the internet via applications such as (Zoom, Microsoft Teams, FaceTime, etc.). Given the nature of work since COVID-19, such remote modalities would enable the tester to perform the needed tests while away from an office setting or to expand the reach of the tests to foreign users. In other words, the tester may perform the muscle response testing regardless of location. These remote modalities expand the reach and scope of services to subjects around the world.

While this disclosure describes uses in the fields of psychology and self-improvement, it will be understood that the method as described herein also has the potential for broader applications in other fields. In education, for example, the method may be used to identify and replace limiting beliefs in students, enhancing their learning abilities and self-confidence. In sports, athletes could benefit from identifying and replacing negative beliefs affecting their performance. Regarding stress management, the method may be adapted to help individuals manage and replace stress-inducing belief systems. For corporate training, the method may be used for personal development and improving interpersonal skills among employees. In the field of addiction rehabilitation, the method could be used to identify and replace negative belief systems vital in addiction recovery. Accordingly, the method of identifying, neutralizing, and replacing negative beliefs within the subconscious mind solves the need for a quick, user-friendly method of improving the mental and emotional well-being of individuals across various domains.

Further, while this disclosure has described the step of showing a mind map to a subject, it will be appreciated that showing the mind map to the subject is not required. Experiments have shown that there is a field of consciousness that connects everyone on earth. This field, referred to as the “Morphic Field” by Dr. Rupert Sheldrake of Cambridge University, contains all the information about everything that exists or that has ever existed. Because of this, the subconscious mind of every individual has access to this field, sometimes referred to as the “database of genius,” and “the database of all that is.” It is clear that every subject has access to this field—via their subconscious mind-and to the mind maps disclosed herein.

Because of this, a subject does not need to see or be shown the mind map prior to a practitioner using the method of identifying negative beliefs with a subject. In other words, the practitioner may ask a series of questions to the subject related to the mind map without the subject having first, consciously, studied the mind map. Accordingly, in some embodiments, the method of identifying negative beliefs comprises asking the subject one or more questions based on a mind map (unknown to the subject's conscious mind), performing muscle response testing to determine the answers to the questions, and identifying the negative belief held within the subject's subconscious mind. Once the negative belief is identified, the method may further comprise neutralizing the negative belief via a magnetic field device and subsequently implanting a positive belief in place thereof, if so indicated by the subject's subconscious mind.

It will be appreciated that systems and methods according to certain embodiments of the present disclosure may include, incorporate, or otherwise comprise properties or features (e.g., components, members, elements, parts, and/or portions) described in other embodiments. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment unless so stated. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. A method of identifying negative beliefs using a mind map, the method comprising:
presenting a mind map to a subject, wherein the mind map comprises one or more categories, one or more subcategories, and one or more sets of negative beliefs;
asking the subject one or more questions based on the mind map;
performing muscle response testing on the subject after each question has been asked; and
identifying a negative belief based on the subject's muscle response testing.

2. The method of claim 1, further comprising swiping a magnetic device over the subject's governing meridian while holding the intention to remove the negative belief.

3. The method of claim 1, further comprising swiping a magnetic device over the subject's governing meridian while holding the intention to install a positive belief.

4. The method of claim 1, further comprising using a monitoring device configured to measure physiological responses of the subject.

5. The method of claim 4, wherein the physiological responses comprise one or more of galvanic skin response, skin conductivity, skin temperature, breathing patterns, electrical brain activity, or heart rate variability.

6. The method of claim 1, wherein the mind map comprises at least seven categories.

7. The method of claim 6, wherein the at least seven categories comprise divine, communication, power, survival, insight, love, and passion.

8. A method of identifying negative beliefs using a mind map, the method comprising:
referencing a mind map, wherein the mind map comprises one or more categories, one or more subcategories, and one or more sets of negative beliefs;
asking the subject one or more questions based on the mind map;
performing muscle response testing on the subject after each question has been asked;
identifying a negative belief based on the subject's muscle response testing; and
swiping a magnetic device over the subject's governing meridian while holding the intention to remove the negative belief.

9. The method of claim 8, further comprising swiping a magnetic device over the subject's governing meridian while holding the intention to install a positive belief.

10. The method of claim 8, further comprising using a monitoring device configured to measure physiological responses of the subject.

11. The method of claim 10, wherein the physiological responses comprise one or more of galvanic skin response, skin conductivity, skin temperature, breathing patterns, electrical brain activity, or heart rate variability.

12. The method of claim 8, wherein the mind map comprises at least seven categories.

13. The method of claim 12, wherein the at least seven categories comprise divine, communication, power, survival, insight, love, and passion.

14. A method of identifying negative beliefs using a mind map, the method comprising:
presenting a mind map to a subject, wherein the mind map comprises a plurality of categories comprising divine, communication, power, survival, insight, love, and passion;
each category of the plurality of categories comprising one or more subcategories, each subcategory comprising one or more sets of negative beliefs;
asking the subject one or more questions based on the mind map;
performing muscle response testing on the subject after each question has been asked;
identifying a negative belief based on the subject's muscle response testing;
swiping a magnetic device over the subject's governing meridian while holding the intention to remove the negative belief; and
swiping the magnetic device over the subject's governing meridian while holding the intention to install a positive belief.

15. The method of claim 14, further comprising using a monitoring device configured to measure physiological responses of the subject.

16. The method of claim 15, wherein the physiological responses comprise one or more of galvanic skin response, skin conductivity, skin temperature, breathing patterns, electrical brain activity, or heart rate variability.

17. The method of claim 14, wherein the mind map comprises at least seven categories.
